(19) United States
(12) Patent Application Publication
(10) Pub. No.: US 2015/0045725 A1
(43) Pub. Date: Feb. 12, 2015

(54) INSUFFLATION AND CO2 DELIVERY FOR MINIMALLY INVASIVE PROCEDURES

(71) Applicant: Vanderbilt University, Nashville, TN (US)

(72) Inventors: Byron F. Smith, Memphis, TN (US); Keith L. Obstein, Nashville, TN (US); Pietro Valdastri, Nashville, TN (US)

(21) Appl. No.: 14/457,676

(22) Filed: Aug. 12, 2014

Related U.S. Application Data
(60) Provisional application No. 61/864,819, filed on Aug. 12, 2013.
(60) Provisional application No. 61/911,094, filed on Dec. 3, 2013.

(51) Int. Cl. A61M 5/168; A61M 5/142

(52) U.S. Cl. CPC A61M 5/16827; A61M 5/142; A61M 5/16881; A61M 2005/006
USPC 604/26

(57) ABSTRACT

Systems and methods are described for providing carbon dioxide insufflation. The system includes a first chamber, a second chamber, and a mixing chamber. The first chamber contains an acid and the second chamber contains a base. The mixing chamber is configured to receive the acid from the first chamber and the base from the second chamber. The mixing chamber is also coupleable to an endoscope and configured to provide an amount of carbon dioxide generated by mixing the acid and the base to the endoscope.

### RELATED APPLICATIONS

This application claims the benefit of U.S. Provisional Application No. 61/864,819, filed Aug. 12, 2013, and U.S. Provisional Application No. 61/911,094, filed Dec. 3, 2013, the entire contents of both of which are incorporated herein by reference.

### STATEMENT REGARDING FEDERALLY SPONSORED RESEARCH OR DEVELOPMENT

This invention was made with government support under grant EEC-0540834 (Subaward No. T5306692601) awarded by the National Science Foundation and grant IIP-1356639 also awarded by the National Science Foundation. The government has certain rights in the invention.

### BACKGROUND

The present invention relates to systems and methods for generating and administering insufflation such as, for example, via a colonoscope, endoscope, or trocar tool channel or through a catheter.

Clinical researchers have been reporting on the benefits of CO₂ insufflation during colonoscopy. Using CO₂ instead of ambient room air for insufflation can reduce procedural and post-procedural pain associated with colonoscopy. However, despite these benefits, a relatively small minority of endoscopists routinely use CO₂ insufflation during traditional colonoscopy. This may be due to a lack of systems and methods of administering CO₂ insufflation that compliments established workflows at a price point where the advantages to the patient outweigh the cost of the system.

To date, commercially available CO₂ insufflators have been defined by compressed gas systems. Because of the inherent danger presented by using a high pressure supply reservoir to provide gas at relatively modest pressures and at a safe flow rate, these systems typically require complicated and expensive electromechanical control units. Such systems can also be require a bulky attachment on the handle of a colonoscope or via an additional catheter which must be run specifically for delivering compressed CO₂. Both such implementations impose additional steps to the clinicians' work-flow and require additional preparation by support staff.

### SUMMARY

Various embodiments described herein provide systems and methods for generating CO₂ and administering said gas via the tool channel of a standard colonoscope or other insufflation delivery device. The systems utilize an effervescent reaction to produce CO₂ in an on-demand fashion that can be manually controlled by a clinician or incorporated into an automated closed-loop system. In some embodiments, the system provides two separate compartments (or chambers) to store reactants until missing is desired to produce an insufflating gas. One or both of the reactants are stored in solution. In some embodiments, the system prevents unwanted byproducts from entering the body cavity. In some embodiments, the system allows the clinician to adjust the position and orientation of the system to suit the clinician's preference in order to promote integration of the system into established work flows.

In one embodiment, the invention provides a carbon dioxide insufflation system including a first chamber, a second chamber, and a mixing chamber. The first chamber contains an acid and the second chamber contains a base. The mixing chamber is configured to receive the acid from the first chamber and the base from the second chamber. The mixing chamber is also coupleable to an endoscope and configured to provide an amount of carbon dioxide generated by mixing the acid and the base to the endoscope.

In some embodiments, the carbon dioxide insufflation system also includes a flow regulator that has a first port coupled to the first chamber, a second port coupled to the second chamber, and a third port coupled to the mixing chamber. The flow regulator configured to provide a desired flow rate of CO₂ to the endoscope by regulating a flow rate of the acid and a flow rate of the base into the mixing chamber.

Other aspects of the invention will become apparent by consideration of the detailed description and accompanying drawings.

### BRIEF DESCRIPTION OF THE DRAWINGS

FIG. 1A is a block diagram of an effervescent insufflation system according to one embodiment.

FIG. 1B is a block diagram of an automated control system for the insufflation system of FIG. 1A.

FIG. 2A is an elevation view of an insufflation system including a double-barreled syringe mechanism.

FIG. 2B is a cross-sectional elevation view of the insufflation system of FIG. 2A.

FIG. 3 is a perspective view of another double-barreled syringe-based implementation of an insufflation system.

FIG. 4 is a perspective view of another insufflation system utilizing pre-measured doses of reactants.

FIG. 5A is an elevation view of an insufflation system including a passive valve for introducing reactants into a mixing chamber.

FIG. 5B is a perspective view of the insufflation system of FIG. 5A.

FIG. 5C is an elevation view of the passive valve component of the insufflation system of FIG. 5A.

FIG. 6 is a perspective view of an insufflation system that utilizes a manually operated pinch valve to control the release of reactants into a mixing chamber.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

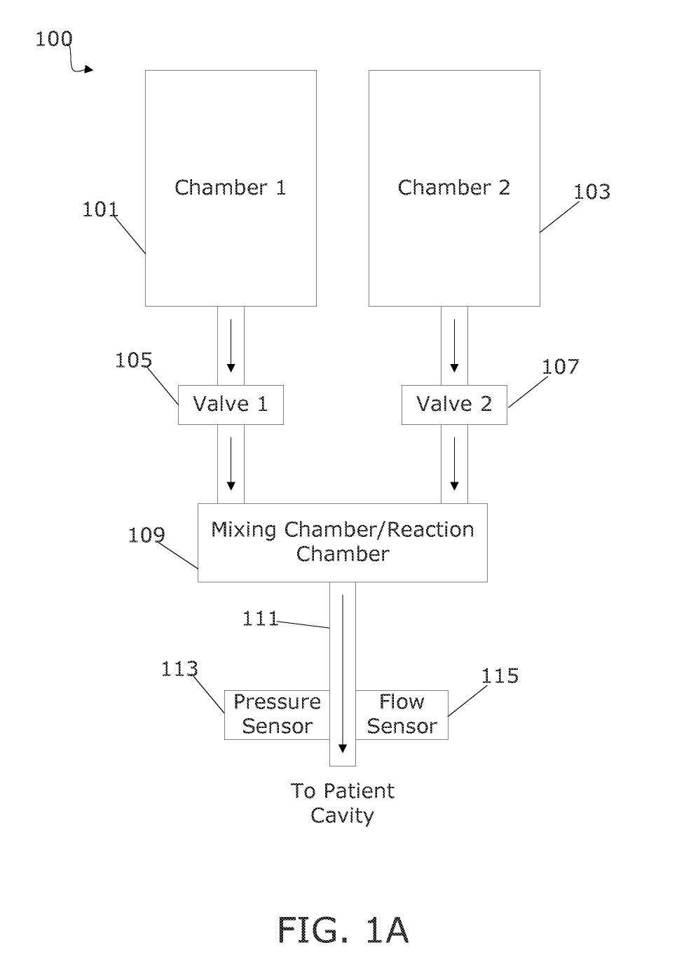

FIG. 1A illustrates the functional components of an effervescent insufflation system 100. The system includes a first chamber 101 that holds a mild acidic solution (e.g, citric acid solution) and a second chamber 103 that holds a mild basic solution (e.g., sodium bicarbonate solution). When CO₂ is required for insufflation, a first valve 105 is opened to release the acidic solution into a mixing/reaction chamber 109 and a second valve is opened to release the basic solution into the mixing/reaction chamber 109. When the acidic solution mixes with the basic solution in the mixing/reaction chamber, the chemical reaction produces CO₂ which is then released through an output tube 111 to the endoscope and used to insufflate the anatomy of a patient.

In the example of FIG. 1A, the output of the mixing/reaction chamber 109 to the output tube 111 is monitored with a pressure sensor 113 and a flow rate sensor 115. As illustrated in FIG. 1B, the output of the pressure sensor 113 and the flow rate sensor 115 are provided to a controller 117. The controller 117 can be implemented, for example, as a microprocessor and a memory stored instructions that are executed by the microprocessor to control the operation of the system. In response to the measurements from the pressure sensor 113 and the flow rate sensor 115, the controller 117 provides an output signal to control the first valve 105 and the second valve 107. Thereby, the controller 117 controls the amount of reactants that are released into the mixing/reaction chamber 109 and regulates the pressure/flowrate of the resultant CO₂ that is delivered through the endoscope for the purpose of insufflation.

The flow of reactants into the mixing/reaction chamber 109 can be controlled by other mechanisms in addition to or instead of the controllable valves 105, 107 illustrated in the example of FIGS. 1A and 1B. For example, some constructions may include an electric pump motor 119 that actively pumps reactants from their respective chambers into the mixing/reaction chamber 109.

The example illustrated in FIGS. 1A and 1B provides a closed-loop automated insufflation system that uses a controller to regulate the amount of CO₂ that is produced by releasing reactants into the mixing/reaction chamber 109. If the pressure sensor indicates that the output pressure is below a target or threshold pressure, the controller 117 releases more reactant to produce more CO₂. Similarly, the controller 117 can control the rate at which the reactants are released into the mixing/reaction chamber 109 to regulate the flow rate of the resultant CO₂ that is delivered through the endoscope.

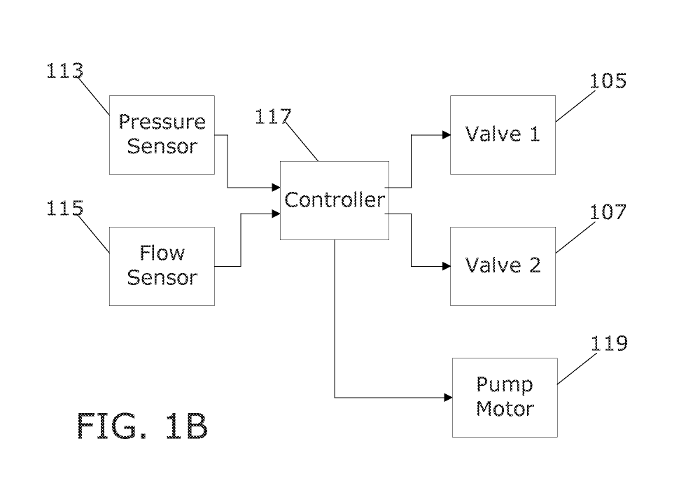

However, in some constructions, the reactants can be manually pumped into the mixing/reaction chamber by a clinician to provide CO₂ in an “on-demand” fashion. FIGS. 2A and 2B illustrate an example of an insufflation system 200 that includes a double-barreled syringe 201 that stores the mild acidic solution in a first barrel and the mild basic solution in the other. When more CO₂ is required, the clinician presses the plunger of the double-barreled syringe 201 to push the reactants into a mixing nozzle 203. The mixing nozzle 203 promotes fast and efficient mixing as the two solutions exit their respective chambers in the double-barreled syringe 201. The mixed reactants then flow into a settling chamber 205 which provides a place for mixed solution to react and also separates the reacting solution from output/exhaust channel 207 such that solid byproducts of the chemical reaction between the acidic and basic solutions are not passed beyond the settling chamber 205. The resultant CO₂ then flows through the exhaust channel 207 that can be plugged directly into the tool channel of a colonoscope, endoscope, or trocar or can be coupled directly to a catheter. The system 200 also includes a mounting bracket 209 that adjustably couples the exhaust channel to the double-barreled syringe 201. This mechanism 209 can be adjusted for positioning and orienting the device relative to the colonoscope, endoscope, or trocar tool channel such that it provides ergonomic access to the device by clinicians in order to promote its integration into established workflows.

Although the example of FIGS. 2A and 2B illustrates a manually operated, syringe-based insufflation system, the system 200 could be modified to include a motor that controls the position and movement of the syringe plunger such that that double-barreled syringe 201 can be incorporated into a closed-loop control system in which CO₂ pressure and flow rate is regulated by a controller (e.g., controller 117 in FIG. 1B).

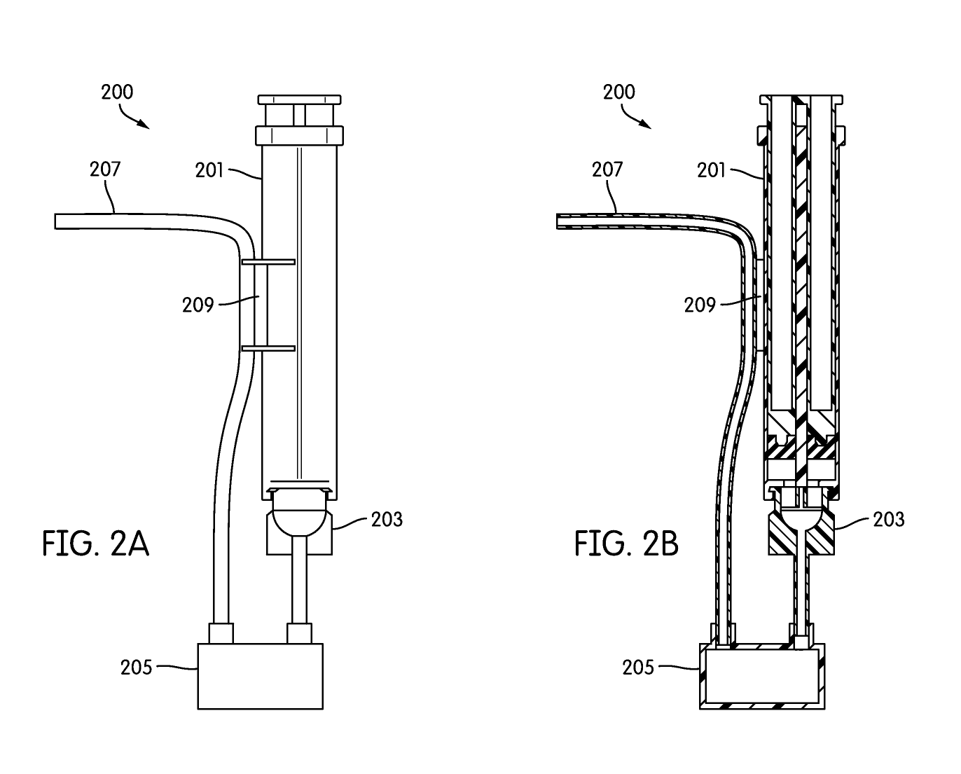

Although the example of FIGS. 2A and 2B illustrates a defined and separate mixing nozzle, the insufflation system can be implemented without a separate mixing nozzle. For example, FIG. 3 illustrates an insufflation system 300 that again includes a double-barreled syringe 301 that stores the acidic solution and the basic solution in separate chambers. When the plunger of the syringe 301 is moved, the reactants are pushed directly into the mixing/settling chamber 303 through two separate openings 305, 307. The exhaust port 309 of the mixing/settling chamber 303 is located above the point where reactants enter the chamber (openings 305, 307) in order to prevent solid byproducts of the chemical reaction from exiting the device through the exhaust port 309. As such, only the produced CO₂ exits the mixing/settling chamber 303 through the exhaust port 309 and is provided through the endoscope, colonoscope, trocar, or catheter for insufflation.

The examples of FIGS. 2A, 2B, and 3 illustrate a double-barreled syringe mechanism where an acid and a base are both stored in solution in separate chambers of the syringe. However, in other constructions, the double-barreled syringe is replaced with a single barrel syringe. The single barrel syringe holds one of the reactants in solution (i.e., an acidic solution or a basic solution) and the other reactant is already provided within the settling chamber as either a solid or a solution.

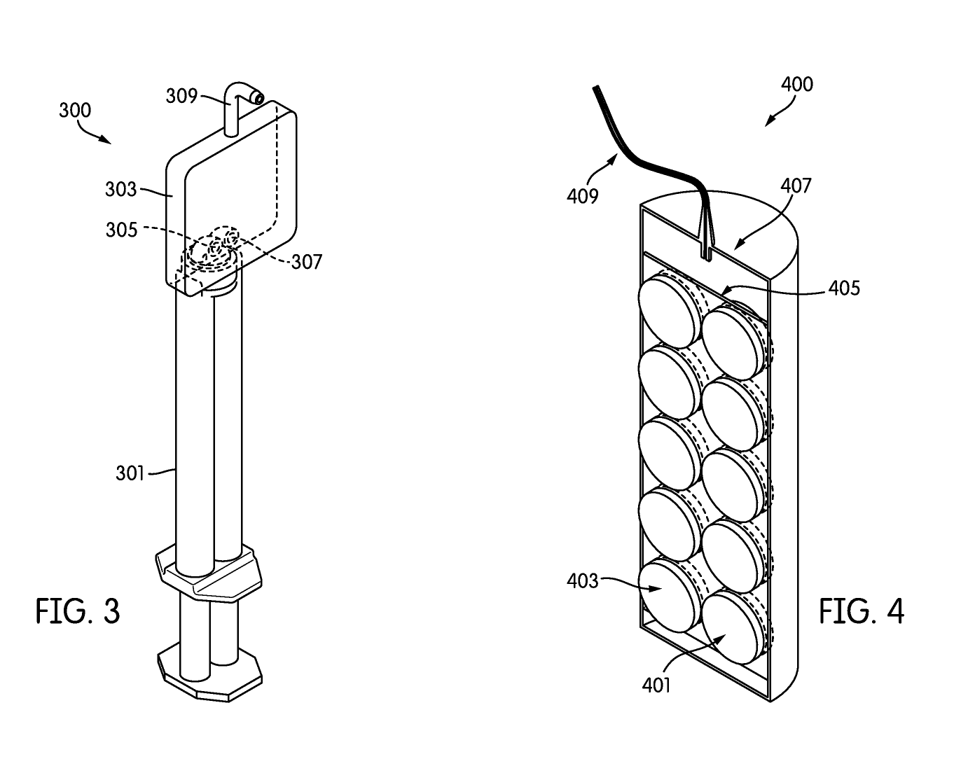

FIG. 4 illustrates yet another example of an insufflation system 400. In this construction, paired masses of reactants are stored in individual compartments that can be popped as necessary. An array of acidic solution masses 401 and basic solution masses 403 are held stationary by a structure 405. When CO₂ is required, a pair of premeasured masses are “popped” releasing the acidic solution and the basic solution into the reacting/settling chamber 407. The reacting/settling chamber 407 is a compliant structure where reactants enter at the bottom of the chamber (i.e., flowing downward when the individual masses are “popped”) and exhaust gas exits from the upper portion of the chamber through exhaust port 409. Again, by positioning the exhaust port above the location at which the acid and the base are mixed, any solid byproducts produced by the chemical reaction are unable to exit the reacting/settling chamber 407 through the exhaust port 409. As such, only the resultant CO₂ flows through the exhaust port 409 to the attached endoscope, colonoscope, or trocar tool channel.

While the example of FIG. 4 has the advantage of requiring low tolerance and, therefore, low cost components, systems that utilize either a single or double-barreled syringe (such as those illustrated in FIGS. 2A, 2B, and 3) have the advantage of analog control which can be provided via visual feedback of the clinician or through the closed-loop electro-mechanical system described above in reference to FIGS. 1A and 1B.

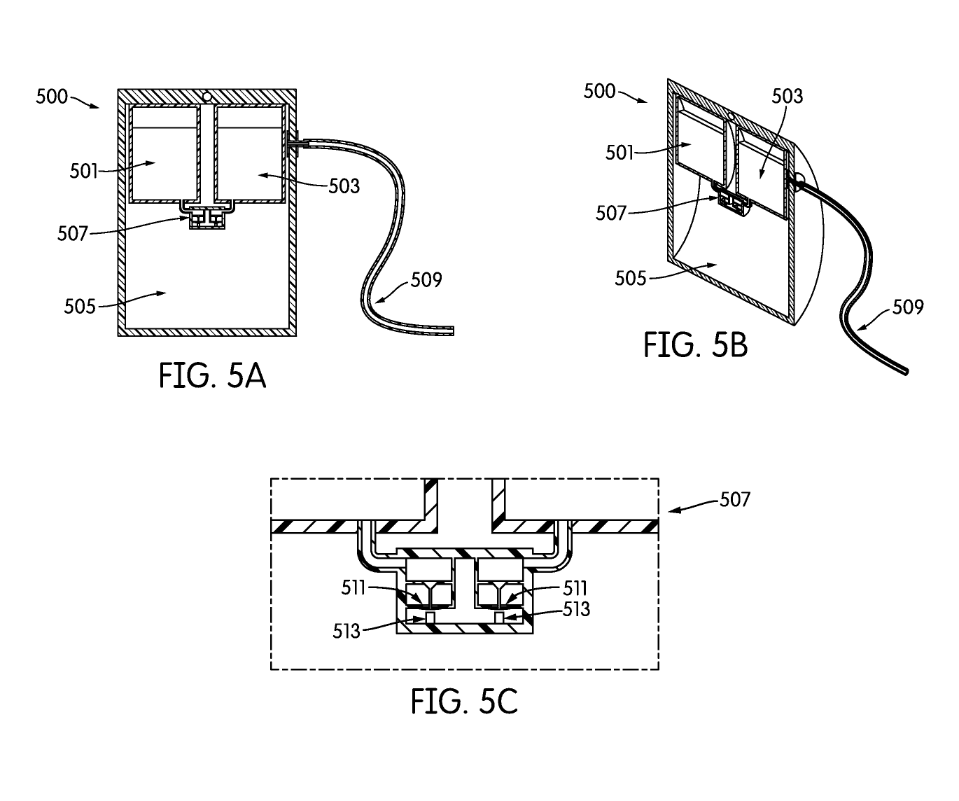

FIGS. 5A, 5B, and 5C illustrate another system 500 that provides the clinician with a steady supply of CO₂ without requiring discrete activation each time the clinician wishes to introduce additional volumes of CO₂. As such, the system can be activated at the onset of a procedure, adjusted to provide a desired flow rate and then left unattended during the duration of the procedure, all the while continuously providing the clinician with the ability to administer CO₂ in a manner that compliments their clinical workflows. Such a insufflation system can access the pneumatic circuit of an endoscope by way of a rinse bottle.

The system 500 is designed such that it can be hunger from an IV-post or similar structure. The system 500 includes three main chambers: a first chamber 501 that holds the acidic solution, a second chamber 503 that holds the basic solution, and a main chamber 505. The reactant chambers 501, 503 are positioned inside the main chamber 505 or otherwise integrated into the structure of the main chamber 505 along the upper edge of the main chamber 505. When the main chamber 505 is hung from an IV support port or the like, gravity initially allows fluid to flow from the reactant chambers 501, 503 into a passive-yet adjustable flow control mechanism 507. As fluid flows through the flow control mechanism 507, it enters a mixing nozzle before falling into the system's main chamber 505. This main chamber allows the mixed solutions to further react while also serving to store the reacted fluids in a manner that ensures that they do not escape through the gas outlet and make their way into the rinse water supply. Because the reactant chambers 501, 503 are compliant in nature, and because they are contained within the main mixing/reaction chamber 505, once the reaction has begun, the pressure generated by the reaction will serve to keep a relatively steady pressure on the acidic and basic solutions. This pressure will ensure that the solutions are fed into the flow control mechanism 507 under greater inlet pressures than might otherwise be achieved using gravity alone.

FIG. 5C provides a detailed view of the non-relieving pressure regulator 507. The regulator 507 includes an orifice and a nozzle connected to a diaphragm 511 for each of the reactant chambers 501, 503. As pressure within the reaction chamber builds, the diaphragm 511 deforms and the resulting displacement causes the nozzle to close the orifice and prevent reactant from entering the mixing/reaction chamber 505. As CO₂ exits the system through the exhaust line 509 and makes its way into the endoscope, pressure within the main mixing/reaction chamber 505 falls and the diaphragm returns to its initial resting position. This causes the nozzle to move away from the seat of the orifice such that the reactant fluid once again flows and CO₂ is generated until the pressure within the mixing/reaction chamber 505 once again reaches the desired level.

A pair of jack screws 513 are positioned below each diaphragm 511 to set the closing pressure of each respective valve. The position of the screw 513 and the size of the orifice can be varied for each respective reactant chamber 501, 503 depending upon the specific reactants that are used and the nature of the specific chemical reaction.

The system 500 illustrated in FIGS. 5A, 5B, and 5C also includes a safety feature to ensure maximum pressure within the system does not rise above a pre-determined level. The upper edge of the main reaction chamber 505 includes a lightly perforated section such that, should the pressure within the main chamber 505 exceed an upper threshold, the main chamber 505 will rupture along the perforation. Although such a rupture would destroy the insufflation system, the replacement cost would be inconsequential when compared to the damage that might result from exposing a patient's colon to unsafe pressurization.

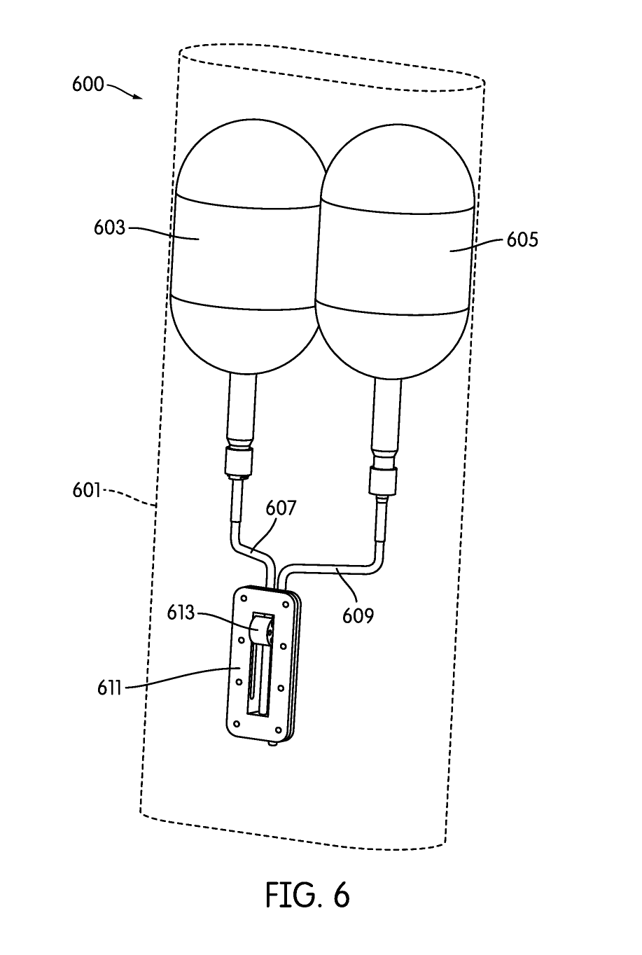

FIG. 6 illustrates another variation of an insufflation system 600. In this example, the system 600 includes a main housing 601 and a pair of reactant chambers 603, 605 positioned within the housing 601. Reactants flow from each respective reactant chamber 603, 605 through a outlet hose 607, 609. The outlet hoses 607, 609 are positioned within a roller-ball pinch valve 611. The pinch valve includes a cylindrical shaped roller 613 mounted on a pair of tracks. When the roller 613 is moved into a first position the outlet hoses 607, 609 are pinched closed such that reactant cannot flow from the reactant chambers 603, 605. When the roller 613, is moved into a second position, the outlet hoses 607, 609 are released and reactants are able to flow into a mixing chamber 601.

The pinch-valve implementation of FIG. 6 can be incorporated into the diaphragm-based valve system of FIGS. 5A, 5B, and 5C. The pinch valve 611 is configured to prevent the release of reactants into the pressure regulator 507. When the pinch valve is closed, no reactants are available and the pressure will drop. However, when then pinch valve is opened, reactants are made available and the passive, pressure regulation is implemented as described above.

Similarly, the roller pinch valve 613 can be coupled to the output hose 509 of the system 500. As such, when the pinch valve 613 is closed, CO₂ does not escape from the main chamber 505 and the resulting pressure causes the diaphragm 511 of the pressure regulator 507 to close. When the pinch valve 613 is opened and CO₂ is released from the main chamber 505, the pressure begins to drop until the pressure regulator 507 allows more reactant to flow into the main chamber and produce more CO₂.

Furthermore, although the examples described above discuss mechanisms that include separate “valves” for each of the reactant chambers, some constructions can utilize a single valve component that controls the flow of reactants from both chambers. For example, the passive pressure regulating diaphragms 511 of FIG. 5C can be replaced with a single pressure regulating diaphragm that obstructs the output of both reactant chambers.

Thus, the invention provides, among other things, systems and methods for effervescent insufflation by controllably releasing an acidic reactant and a basic reactant into a mixing chamber to produce CO₂. Various features and advantages of the invention are set forth in the following claims.

What is claimed is:

1. A carbon dioxide insufflation system comprising:
a first chamber containing an acid;
a second chamber containing a base; and
a mixing chamber configured to receive the acid from the first chamber and the base from the second chamber, the mixing chamber coupleable to a device for insufflation delivery and configured to provide an amount of carbon dioxide (CO₂) generated by mixing the acid and the base to the device for insufflation delivery.

2. The carbon dioxide insufflation system of claim 1, further comprising a flow regulator including a first port coupled to the first chamber, a second port coupled to the second chamber, and a third port coupled to the mixing chamber, the flow regulator configured to provide a desired flow rate of CO₂ to the device for insufflation delivery by regulating a flow rate of the acid and a flow rate of the base into the mixing chamber.

3. The carbon dioxide insufflation system of claim 2, wherein the first chamber and the second chamber are positioned within the mixing chamber.

4. The carbon dioxide insufflation system of claim 3, wherein the first chamber and the second chamber are compliant, and wherein a steady pressure is applied to the first chamber and the second chamber during reaction of the acid and the base in the mixing chamber.

5. The carbon dioxide insufflation system of claim 4, wherein the steady pressure applied to the first chamber and the second chamber regulates flow of the acid and the base are fed into the flow regulator.

6. The carbon dioxide insufflation system of claim 2, wherein the flow regulator includes a first nozzle removably coupled to the first port, a first diaphragm, a second nozzle removably coupled to the second port, and a second diaphragm.

7. The carbon dioxide insufflation system of claim 6, wherein the first diaphragm deforms to cause the first nozzle to close the first port and the second diaphragm deforms to cause the second nozzle to close the second port when pressure inside the mixing chamber increases to a predetermined level.

8. The carbon dioxide insufflation system of claim 6, wherein the first diaphragm resumes a resting position to cause the first nozzle to open the first port and the second diaphragm resumes a resting position to cause the second nozzle to open the second port when pressure inside the mixing chamber decreases to a predetermined level.

9. The carbon dioxide insufflation system of claim 1, wherein the acid is citric acid.

10. The carbon dioxide insufflation system of claim 9, wherein the base is sodium bicarbonate.

11. The carbon dioxide insufflation system of claim 9, wherein the citric acid is in solid form.

11. The carbon dioxide insufflation system of claim 1, wherein the base is sodium bicarbonate.

12. The carbon dioxide insufflation system of claim 11, wherein the sodium bicarbonate is in solution.

13. The carbon dioxide insufflation system of claim 1, further comprising a double-barreled syringe, wherein a first barrel of the syringe includes the first chamber and a second barrel of the syringe includes the second chamber.

14. The carbon dioxide insufflation system of claim 1, further comprising:
a pressure sensor configured to monitor a pressure of the mixing chamber; and
a controller configured to
receive a signal from the pressure sensor indicative of the pressure of the mixing chamber,
compare the pressure of the mixing chamber to a pressure threshold, and
cause the acid and the base to flow into the mixing chamber when the pressure of the mixing chamber is below the pressure threshold.

15. The carbon dioxide insufflation system of claim 14, further comprising a controllable valve between the first chamber and the mixing chamber, and wherein the controller is configured to cause the acid to flow into the mixing chamber by opening the controllable valve.

16. The carbon dioxide insufflation system of claim 14, further comprising a controllable pump, and wherein the controller is configured to cause the acid to flow into the mixing chamber by operating the controllable pump.

17. The carbon dioxide insufflation system of claim 1, further comprising:
a flow rate sensor configured to monitor a flow rate of CO₂ from the mixing chamber; and
a controller configured to
receive a signal from the flow rate sensor indicative of the flow rate,
compare the flow rate to a flow rate threshold, and
cause the acid and the base to flow into the mixing chamber when the flow rate is below the flow rate threshold.

18. The carbon dioxide insufflation system of claim 1, wherein the device for insufflation delivery includes at least one selected from the group consisting of an endoscope, a colonoscope, a trocar, and a catheter.
